SEQUENCE LISTING (17 sequences)

SEQ ID NO: 1 - DNA, 1026 bp, synthetic construct
  source 1..1026: mol_type = other DNA
atggaggcagagaagaaacattcatcagccgtttctaatgtgggagcttgggcaatgaacgttattagctccgtcgggttaatcatggtcaacaagcaactcatgtcccctaccggtcatgccttcgcttttgcgacaacattaactgggttccacttttgtacaactgccttaatcggtttggtgtcaaatgccactggttacacaacaaaaaagaatgttcctttgtgggagcttctttggttctcggttgttgccaatacttcaattaccgcgatgaacttgagcctcatgttgaattcagttggattttatcagatttcaaagctaagtatgattcctgttgtttgctttctggaatggctcctcaatggtaaacattactcgagtaaagtcaagatggcagtggtggtggtggtggtgattggtgtaggtgtttgtacagttactgatgtaaaaattaattcgcatggatttctttgtgcctgtgtagctgtcctatctacctccttacaacaaatttcaattgggtccttacagaagaagtactcaataggatcttttgaactgcttagccaaacagctccaatccaagctttatctcttttgttgcttggtccattcgttgattacttcctcactggcaagttactagcaagttataagatctcctcagctgcattttttttcatagtactgtcatgttccttagctgttttctgcaacataagccagtacctgtgcattggacgattctcggcggtgtctttccaagtgctaggacacatgaaaacagtatgcgtgttgatattaggatggatgctgtttgactcagagctgacattgaaaaacatactagggatgagcatagctgtgaatgggatgataatatatagttgggcagttgagggagataagccaatgcactaccgaaaagatgctacatcagatgtaaagctactgataaagcaagtggacggctcatcactactcaaggacgttgagcttggcaaatctcaaggatag

SEQ ID NO: 2 - protein, 1023 aa, synthetic construct
METGLUALAGLULYSLYSHISSERSERALAVALSERASNVALGLYALATRPALAMETASNVALIIESERSERVALGLYLEUIIEMETVALASNLYSGLNLEUMETSERPROTHRGLYHISALAPHEALAPHEALATHRTHRLEUTHRGLYPHEHISPHECYSTHRTHRALALEUIIEGLYLEUVALSERASNALATHRGLYTYRTHRTHRLYSLYSASNVALPROLEUTRPGLULEULEUTRPPHESERVALVALALAASNTHRSERIIETHRALAMETASNLEUSERLEUMETLEUASNSERVALGLYPHETYRGLNIIESERLYSLEUSERMETIIEPROVALVALCYSPHELEUGLUTRPLEULEUASNGLYLYSHISTYRSERSERLYSVALLYSMETALAVALVALVALVALVALVALIIEGLYVALGLYVALCYSTHRVALTHRASPVALLYSIIEASNSERHISGLYPHELEUCYSALACYSVALALAVALLEUSERTHRSERLEUGLNGLNIIESERIIEGLYSERLEUGLNLYSLYSTYRSERIIEGLYSERPHEGLULEULEUSERGLNTHRALAPROIIEGLNALALEUSERLEULEULEULEUGLYPROPHEVALASPTYRPHELEUTHRGLYLYSLEULEUALASERTYRLYSIIESERSERALAALAPHEPHEPHELLEVALLEUSERCYSSERLEUALAVALPHECYSASNIIESERGLNTYRLEUCYSILEGLYARGPHESERALAVALSERPHEGLNVALLEUGLYHISMETLYSTHRVALCYSVALLEUIIELEUGLYTRPMETLEUPHEASPSERGLULEUTHRLEULYSASNIIELEUGLYMETSERIIEALAVALASNGLYMETIIEIIETYRSERTRPALAVALGLUGLYASPLYSPROMETHISTYRARGLYSASPALATHRSERASPVALLYSLEULEUIIELYSGLNVALASPGLYSERSERLEULEULYSASPVALGLULEUGLYLYSSERGLNGLY

SEQ ID NO: 3 - DNA, 2500 bp, synthetic construct
  source 1..2500: mol_type = other DNA
tatcacattaaatgaactcgcttctggcagatgaagttttagttttgtcttcaggatcctgctacgtctagcatgcttgaaagtttgtccaatcctcagcaaaaagaccagattgaggaaagaatggcacgtatcaaagaggatccatcattgaaacatattctagaagaaatagagacaggtggtcccgcagccatgatgaggtgggtgatcaaattactagtagcatcatagtattatagtgttgtgggcacaacagtttcttctttagaacgtatgtagatgtccgcatttagctatttttcactaaattaataacagaaaggcttctggattaggtactggaatcataaagaagttctccagaagttgggtgaagcaatgggtcttgcagattcaggagatgcagccatttctgttggtaaggctgcagccgatgaagatgatgaagtagggaatgaggatgagtcaattgtacatcattgtgctagtgttggtgatgttgaggtaggacagagtgaatttttgtacatgtatttttcgcctttgatagataatctaaaaagtctcgactgtatttgtggtttagggtctgaaaactgcactagcctctggtgctgacaaggatgaagaagatgcagaggggaggacggcattacattttgcatgtggttatggcgaggtatgataattttataattgaaatctgggtagtccctactgctgcattgcctgactaatgaagtagttttgtctatcgaggacaaacttgtgtggaaaatctgatgggtttaataactgattgttacaagtattcaatactagtctcaaatgccggtggacacctctagtctagtagtttaggagagtttgtaggagattgggttattgggctagttttaggttatgtttcttagaaaagatttcaggcaaagggatagattccatgtcatgttttttacccgggggtaaagctaatacactagtgattttattattgtagatgaaatgtgcacaaatcctagttgaagctggagcaagagtggatgctttggacaagaataagaatactgcacttcattatgcagctggttatggaaggaaagactgtgtggcccttctactggagaacggggctgctgtgtaagtaaacaaactcgttgggtaaccattgatatgtatattgacttgtttatgtaatttatttattttattttattttttatgtcagcacactccaaaacatggatggcaagacccccattgaagtagccaagctgaacaatcagcatgaggtagtaaagttgttggagaaagatgcgtttttgtgagttgggagggggcattgcgcccatgaattaaattgtacctcgaggaaaagggaatgggatgaaggtaaagtggtgaactcaaaacaataaataaataaaatatactgaatacagttatatgaacgaaagatctgtagatgaagactggtagacaacaacaatgtttattgtgttttgttaatcacagtttcatcacttaaattctagaattattgtataatgcttaattgataaattaatctaattgtaatttgttgggcttaatgccaaaaaagttcggcttaaaagcaatgttttggaaattagattggtggtctaatcagtcaaaatgtatcggtcgtttgtaaataatttatgtaaatataagatgaaaatgtgaccaaaaaagagataagatatcaacaataaatgtgacaaacaaattataatagtcattttgaccatacataaatggtctacatttatcttgtcacggttgattatttaaggagtatatttatcttctagatagatgatgataagattaatatagattaaacaaagtttacttgtgcttcatgcaaagaaaagacatttcatgtgcgacgtatacatatacctacagaatttatagttgacaaaatgttgtcgttgaaggaagctatataagtgatgagataaatttaaataaaaagaggaaattcaaaggaaagaacaaaaaaggtgtgagagaaaaaagaaaagggaggtgggggggaggccaagaagctaccatctgccaaactggagtaaaaataggatccacactcttcacaccggtttcttactttctatttttatttttcacaaatttaaaaagagattagggcaattgacattttccaaatactttaacaatcgatgatctactactcaccatgtcattcgttcttgttcaaagagaattaaatcgtaaatatataaatttatgtaactattaataaaaaaagaaaattggaacataattcggatccaactactgtatataaagtcgtgatcgttcaaaatttacatgtttcagattttctcgtatatcttgctctgagctgcctctgcgtgcgggggcaggatcggagattgtttggtcggcgaagtttcagatctgtctttgtagtccaatcgactggtaatattcgatttggattgcgaca

SEQ ID NO: 4 - DNA, 23 bp, synthetic construct
  source 1..23: mol_type = other DNA
ccaagctcccacattagaaacgg

SEQ ID NO: 5 - DNA, 23 bp, synthetic construct
  source 1..23: mol_type = other DNA
cattccagaaagcaaacaacagg

SEQ ID NO: 6 - DNA, 24 bp, synthetic construct
  source 1..24: mol_type = other DNA
acacaggagcgtttatataagcga

SEQ ID NO: 7 - DNA, 21 bp, synthetic construct
  source 1..21: mol_type = other DNA
tggtttgttggtcgccgttag

SEQ ID NO: 8 - DNA, 20 bp, synthetic construct
  source 1..20: mol_type = other DNA
gggcaggatcggagattgtt

SEQ ID NO: 9 - DNA, 19 bp, synthetic construct
  source 1..19: mol_type = other DNA
gcatcggaacccaacagga

SEQ ID NO: 10 - DNA, 20 bp, synthetic construct
  source 1..20: mol_type = other DNA
ggttctcggttgttgccaat

SEQ ID NO: 11 - DNA, 21 bp, synthetic construct
  source 1..21: mol_type = other DNA
agataggacagctacacaggc

SEQ ID NO: 12 - DNA, 55 bp, synthetic construct
  source 1..55: mol_type = other DNA
gaaacattcatcagccgtttctaatgtgggagcttgggcaatgaacgttattagc

SEQ ID NO: 13 - DNA, 55 bp, synthetic construct
  source 1..55: mol_type = other DNA
ctttgtaagtagtcggcaaagattacaccctcgaacccgttacttgcaataatcg

SEQ ID NO: 14 - DNA, 50 bp, synthetic construct
  source 1..50: mol_type = other DNA
gaaacattcatcagccgtttgtgggagcttgggcaatgaacgttattagc

SEQ ID NO: 15 - DNA, 55 bp, synthetic construct
  source 1..55: mol_type = other DNA
aagctaagtatgattcctgttgtttgctttctggaatggctcctcaatggtaaac

SEQ ID NO: 16 - DNA, 55 bp, synthetic construct
  source 1..55: mol_type = other DNA
ttcgattcatactaaggacaacaaacgaaagaccttaccgaggagttaccatttg

SEQ ID NO: 17 - DNA, 53 bp, synthetic construct
  source 1..53: mol_type = other DNA
aagctaagtatgattcctgttttgctttctggaatggctcctcaatggtaaac

(19) United States
(12) Patent Application Publication
(10) Pub. No.: US 2025/0270585 A1
(43) Pub. Date: Aug. 28, 2025

(54) Recessive Photoperiod-Sensitive Genic Male Sterile Gene Ghpsm5 and Use Thereof in Cotton

(71) Applicant: Institute of Cotton Research of CAAS, Anyang (CN)

(72) Inventors: Shuli Fan, Anyang (CN); Chaojun Zhang, Anyang (CN); Qifeng Ma, Anyang (CN); Kaikai Qiao, Anyang (CN)

(21) Appl. No.: 18/304,802

(22) PCT Filed: Dec. 7, 2022
(86) PCT No.: PCT/CN2022/137175

§ 371 Date: May 8, 2025

(30) Foreign Application Priority Data
Nov. 22, 2022 (CN) 202211463130.X

(51) Int. Cl. C12N 15/82; C12N 9/22; C12N 15/11; C12Q 1/6895

(52) U.S. Cl. CPC C12N 15/8289; C12N 9/226; C12N 15/11; C12N 15/8205; C12Q 1/6895; C12N 2310/20; C12Q 2600/13

(57) ABSTRACT

The present disclosure discloses a recessive photoperiod-sensitive genic male sterilie (RPGMS) gene Ghpsm5 and use thereof in cotton, and relates to the technical field of genetic engineering and genetic breeding of agricultural crop. The present disclosure provides a cotton RPGMS gene Ghpsm5. Changing the sequence of Ghpsm5 gene in normal cotton plants may make Ghpsm5 unable to be expressed normally, which leads to male sterility of cotton under long sunshine conditions with a sunshine duration greater than or equal to 12.5 hours, restored male fertility under short sunshine conditions with a sunshine duration less than or equal to 12.0 hours, and failures of self-crossing and boll-setting under a condition with a sunshine duration greater than 12.0 hours but less than 12.5 hours where the cotton may still be used for hybrid seed production. Changing Ghpsm5 has no effect on female fertility. The gene may be used for cotton breeding, and cotton RPGMS lines may be prepared by inhibiting the expression of this gene through biotechnologies.

This application claims the priority of Chinese Patent Application No. 202211463130.X, entitled “Recessive photoperiod-sensitive genic male sterile gene Ghpsm5 and use thereof in cotton” filed with the China National Intellectual Property Administration on Nov. 22, 2022, which is incorporated herein by reference in its entirety.

### TECHNICAL FIELD

The present disclosure belongs to the technical field of genetic engineering and agricultural crop genetic breeding technology, in particular to a recessive photoperiod-sensitive genic male sterile (RPGMS) gene Ghpsm5 and use thereof in cotton.

### BACKGROUND ART

Cotton is one of the major crops in the world, which is currently cultivated in more than 70 countries. China, India and the United States are the major cotton producers. The main task of cotton scientific research is to improve the yield and quality of cotton. Heterosis is one of the most successful biological phenomena applied in agriculture, and cotton is also a cash crop with obvious heterosis. The hybrid cotton has the advantages as to nutritional growth, yield and resistance to stresses such as high temperature, humidity, drought, leanness and disease, so the application of hybrids has become an important means for cotton breeding. The United States began the research on the utilization of cotton heterosis in the 1940s. It was not until 1970s that the cytoplasmic male sterile line with Hacknessy cotton was developed, and the three-line method was realized. The breeding of hybrid cotton in China has been developing rapidly. The three-line method using cytoplasmic male sterile lines, the two-line method using recessive nuclear male sterile lines and the hand-emasculation assisted pollination method have been applied. The utilization of hybrid cotton in China reached its peak in 2007. Hybrid cotton accounted for more than 90% of the cotton area in the Yangtze River Basin. However, the hybrid seeds are mainly produced by hand-emasculation assisted pollination. After 2007, the cost of hybrid cotton seeds rose sharply with the increase of labor costs, resulting in a rapid reduction of hybrid cotton planting area. The successive success of the “three-line method” and “two-line method” hybrid rice has produced huge social and economic benefits, while the selection of restorer lines of cotton “cytoplasmic male sterile line” is difficult, and there is no excellent combination of advantages. 50% of fertile plants have to be pulled out during seed production of cotton genic male sterile line, and the yield of seed production is limited, so the production of cotton hybrid is still dominated by hand-emasculation. The preparation of a “dual-use line” material that can be used as a sterile line and propagate itself is the key technology for the application of cotton hybrids, and is also a worldwide challenge.

There are many kinds of genic male sterile lines found in cotton. Up to now, 17 different types of genic male sterile lines have been found, including 9 recessive genic male sterile lines, namely ms1, ms2, ms3, ms5ms6, ms8ms9, ms13, ms14, ms15 and ms16, and 8 dominant genic male sterile lines, namely Ms4, Ms7, Ms10, Ms11, Ms12, Ms17, Ms18 and Ms19. Among the 17 male sterile lines, 12 were found in upland cotton, and 5 were found in sea island cotton. The lines ms2 and Ms4 are completely sterile, ms1 and ms3 are only partially sterile, and ms8ms9 shows no anther dehiscence. Molecular markers located on chromosome are found in ms5, ms8 and ms9. The research on genic male sterile gene is in the stage of searching for markers, and there are no patent and report on the above related genes.

The institute of cotton research of Chinese Academy Agricultural Sciences developed a veriscent cotton photoperiod-sensitive genic male sterile (PGMS) mutant Zhong9106 using the space mutation breeding technology, which is male sterile under long sunshine conditions with an illumination period of more than 13.5 hours, and fertile under short sunshine conditions with an illumination period of less than 13.5 hours and a daily average temperature of more than or equal to 21.5° C. Zhang Chaojun obtained a recessive photoperiod-sensitive genic male sterile mutant psm4 (ZL201810132189.8) by culturing a variety of tissues from cotton material W10 and identified molecular markers related to the RPGMS trait (ZL202010869171.3). The gene Ghpsm5, which controls the RPGMS of cotton, was identified by verifying the gene function of the molecular marker associated intervals. The RPGMS material ps201 was obtained by editing the Ghpsm5 gene.

### SUMMARY

An objective of the present disclosure is to provide a RPGMS gene Ghpsm5 and use thereof in cotton. Based on the gene and/or the promoter thereof, RPGMS materials of cotton or other plants are prepared by making use of plant fertility changes included by photoperiod.

The present disclosure provides a cotton RPGMS gene Ghpsm5. The amino acid sequence of the protein encoded by the RPGMS gene Ghpsm5 includes the sequence set forth in SEQ ID NO:2, or the amino acid sequence that has an identity of more than 75% with the amino acid sequence set forth in SEQ ID NO:2.

The present disclosure also provides a cotton RPGMS gene Ghpsm5. The nucleotide sequence of the RPGMS gene Ghpsm5 includes the nucleotide sequence set forth in SEQ ID NO:1, or the nucleotide sequence that encodes the amino acid sequence of a derivate protein with the function of regulating anther dehiscence obtained by replacing and/or deleting and/or adding one or more amino acid residues of SEQ ID NO:2.

The present disclosure also provides a promoter regulating the expression of the cotton RPGMS gene Ghpsm5. The nucleotide sequence of the promoter is set forth in SEQ ID NO:3.

The present disclosure also provides a use of the cotton RPGMS gene Ghpsm5 or the promoter in preparing a PGMS plant material.

In one embodiment, under a condition that the sunshine duration is greater than or equal to 12.5 hours, the RPGMS plant material has abnormal pollen development and no anther dehiscence, resulting in male sterility. Under a condition that the sunshine duration is less than or equal to 12.0 hours, the pollen development and the anther dehiscence are normal, and the male fertility is restored. Under a condition that the sunshine duration is greater than 12.0 hours but less than 12.5 hours, some anthers close to the base of flowers occasionally crack and disperse pollens with normal vitality but small numbers (generally less than 5 anthers), which make it difficult for the cotton to self-cross and set bolls, but may still be used for hybrid seed production. The RPGMS cotton material that blooms in long sunshine may be transformed into male fertile after 15-18 days of short sunshine treatment. The specific transformation time is affected by the development speed of buds. The transformation time of Ghpsm5 gene editing material ps201 is 17 days when it is treated in August in Anyang city: The RPGMS cotton material that blooms in short sunshine and is normally fertile, may be transformed into male sterile after 15-18 days of long sunshine treatment. The specific transformation time is affected by the development speed of buds. The transformation time of ps201 is 17 days when it is treated in August in Anyang city.

The present disclosure also provides a method for preparing a RPGMS plant material, including regulating and/or changing an activity and/or expression of the cotton RPGMS gene Ghpsm5.

In one embodiment, a method for the regulating and/or changing includes one or more of gene editing, RNAi, antisense RNA and DNA methylation.

In one embodiment, a method for the regulating and/or changing includes constructing an expression vector by using the promoter through genetic engineering to express the protein, RNA and/or DNA sequence that can affect the development of male organs.

In one embodiment, the male organ includes anthers.

The present disclosure also provides a use of the RPGMS material in plant breeding.

Beneficial effects: The present disclosure provides a cotton RPGMS gene Ghpsm5. The sequence of Ghpsm5 gene in normal cotton plants is changed, so that Ghpsm5 gene cannot be expressed normally. Under a condition that the sunshine duration is greater than or equal to 12.5 hours, the pollen of the cotton is inactive and has no anther dehiscence, resulting in male sterility. Under a condition that the sunshine duration is less than or equal to 12.0 hours, the pollen development and the anther dehiscence are normal, and the male fertility is restored. Under a condition that the sunshine duration is greater than 12.0 hours but less than 12.5 hours, some anthers close to the base of flowers may crack and disperse pollens with normal vitality but small numbers (generally less than 5 anthers), which make it difficult for the cotton to self-cross and set bolls, but may still be used for hybrid seed production.

Changing the Ghpsm5 gene has no effect on female fertility, and Ghpsm5 has no relationship with the low-temperature yellowing of psm1. Therefore, the gene may be used for cotton breeding, and cotton RPGMS lines may be prepared by inhibiting the expression of this gene through biotechnologies.

### BRIEF DESCRIPTION OF THE DRAWINGS

In order to explain the embodiments of the present disclosure or the technical solutions in the prior art more clearly, the following will briefly introduce the drawings needed in the embodiments. It is obvious that the drawings in the following description are only some embodiments of the present disclosure. For those skilled in the art, other drawings can also be obtained from these drawings without an inventive step.

FIG. 1 is a schematic diagram of plant CRISPR/Cas9-Ghpsm5 vector:

FIG. 2 shows a process of cotton genetic transformation CRISPR/Cas9-Ghpsm5 (A) and photos of each growth and development stage (B), in which a is hypocotyl segment, b and c are early callus, d is late callus, e is embryogenic callus, f: globular embryo, g is heart-shaped embryo, h is torpedo embryo, i is cotyledon embryo, and j is regenerated cotton seedling: the scale of a, b, c, d and e is 5 mm, the scale of f, g, h, i and j is 1 mm:

FIG. 3 shows the expression and editing site analysis results of the cotton RPGMS line ps201 obtained by gene editing: a-d are male fertile plants, anthers and stigma on the day of flowering, normal pollen dispersal anther and activity detection of pollen grains (active) of ps201 under the Southern winter breeding conditions at the south breeding base of the institute of cotton research of Chinese Academy Agricultural Sciences in Yacheng, Hainan Province, respectively: e-h are plants (leaves removed manually), anthers and stigma on the day of flowering, uncracked anther, activity detection of pollen grains (inactive) extracted manually from ps201 under normal male sterile conditions in the experimental field of the institute of cotton research of Chinese Academy Agricultural Sciences in Anyang, Henan Province, respectively:

FIG. 4 shows an editing site of Ghpsm5 gene editing target 1 of the RPGMS plant.

FIG. 5 shows an editing site of Ghpsm5 gene editing target 2 of the RPGMS plant.

### DETAILED DESCRIPTION OF THE EMBODIMENTS

The present disclosure provides a cotton RPGMS gene Ghpsm5. The amino acid sequence of the protein encoded by the RPGMS gene Ghpsm5 includes the sequence set forth in SEQ ID NO:2, or the amino acid sequence that has an identity of more than 75% with the amino acid sequence set in SEQ ID NO:2.

In the present disclosure, the RPGMS material psm5 is mainly used. Closely linked molecular markers of this trait are obtained through genome sequencing, association analysis and molecular marker research on the hybrid offspring of psm5 and W10. The cotton RPGMS gene is obtained through the functional study of candidate genes in the marker region, and named as Ghpsm5. Changing the sequence of Ghpsm5 gene in normal cotton plants may make Ghpsm5 unable to be expressed normally. Under a condition that the sunshine duration is greater than or equal to 12.5 hours, the pollen of the cotton is inactive and has no anther dehiscence, resulting in male sterility. Under a condition that the sunshine duration is less than or equal to 12.0 hours, the pollen development and the anther dehiscence are normal, and the male fertility is restored. Under a condition that the sunshine duration is greater than 12.0 hours but less than 12.5 hours, some anthers close to the base of the flower may crack and disperse pollens with normal vitality but small numbers (generally less than 5 anthers), which make it difficult for the cotton to self-cross and set boll, but may still be used for hybrid seed production. Changing the Ghpsm5 gene has no effect on female fertility, and Ghpsm5 has no relationship with the low-temperature yellowing of psm1. The W10 somatic regeneration plant and various mutants, such as psm5, involved in the present disclosure have been publicized in the article (Breeding of the photosensitive male sterile line psm5 in cotton and the pattern of fertility transformation: Research on Creation and Characteristics of photoperiod Sensitive Genetic Male Sterility Mutant psm4), and the author promises to distribute them to the public within 20 years from the application date.

The term “identity” in the present disclosure refers to the sequence similarity with natural nucleic acid sequence. The term “identity” involves DNA molecules, cDNA molecules or RNA molecules of the nucleotide sequences set forth in SEQ ID NO:1 and/or SEQ ID NO:3, and/or protein composed of amino acid residues set forth in SEQ ID NO: 2, and/or nucleotide sequences with 75% or higher, or 85% or higher, or 90% or higher, or 95% or higher identity thereto. Identity may be evaluated with naked eyes or computer software. When a computer software is used to evaluate the identity between related sequences, the identity between two or more sequences may be expressed as a percentage (%).

The present disclosure also provides a cotton RPGMS gene Ghpsm5, where a nucleotide sequence of the RPGMS gene Ghpsm5 includes the nucleotide sequence as set forth in SEQ ID NO:1, or the nucleotide sequence of a derivate protein with the function of regulating anther dehiscence obtained by replacing and/or deleting and/or adding one or more amino acid residues set forth in SEQ ID NO:2. The cotton RPGMS gene Ghpsm5 is isolated from Gossypium hirsutum.

The present disclosure also provides a promoter that regulates the expression of the cotton RPGMS gene Ghpsm5. The nucleotide sequence of the promoter is set forth in SEQ ID NO:3.

In particular, the sequence having 75% or higher identity with the nucleotide sequence of the Ghpsm5 gene or/and the promoter and encoding the protein set forth in SEQ ID NO:2 is derived from the nucleotide sequence of the present disclosure and is equivalent to the sequence of the present disclosure.

The present disclosure also provides a use of the cotton RPGMS gene Ghpsm5 or the promoter in the preparation of RPGMS plant material.

In the present disclosure, manipulations may be at the DNA level or RNA level to abolish the ability of the Ghpsm5 gene to be completely expressed and/or to be translated into proteins. Alternatively, the promoter can be manipulated at the DNA level to deprive its ability to initiate the expression of downstream genes, thus changing the expression of Ghpsm5 gene. Therefore, a RPGMS material of cotton or other plants is created. In normal field cultivations, under a condition that the sunshine duration is greater than or equal to 12.5 hours, the RPGMS material of the plant has abnormal pollen development and no anther dehiscence, resulting in male sterility. Under a condition that the sunshine duration is less than or equal to 12.0 hours, the pollen development and the anther dehiscence are normal, and the male fertility is restored. Under a condition that the sunshine duration is greater than 12.0 hours but less than 12.5 hours, some anthers close to the base of flowers occasionally crack and disperse pollens with normal vitality but small numbers (generally less than 5 anthers), which make it difficult for the cotton to self-cross and set bolls, but may still be used for hybrid seed production.

The present disclosure also provides a method for preparing a RPGMS plant material, including regulating and/or changing an activity and/or expression of the cotton RPGMS gene Ghpsm5.

A method for the regulating and/or changing includes one or more of gene editing, RNAi, antisense RNA and DNA methylation, and preferably further includes construction of an expression vector using the promoter through genetic engineering method to express the protein, RNA and/or DNA sequences that may affect the development of a male organ. The male organ of the present disclosure preferably comprises anthers.

1. In the present disclosure, the Ghpsm5 gene and/or the promoter sequence are changed by using at least one of the methods including gene editing, RNAi, antisense RNA, DNA methylation, etc., thus changing the activity and/or expression of the Ghpsm5 gene. Therefore, an RPGMS material of cotton or other plants is created. The nucleotide sequence of the RPGMS gene Ghpsm5 comprises any one of a)-d): a) a DNA molecule or cDNA molecule encoded by SEQ ID NO: 1: b) a cDNA molecule or genomic DNA molecule encoding the protein set forth in SEQ ID NO:2 and having an identity of 75% or more with the nucleotide sequence set forth in SEQ ID NO:1: c) a cDNA molecule or genomic DNA molecule encoded by SEQ ID NO:1 and Hybridizing with the nucleotide sequence of SEQ ID NO:1 under strict conditions: d) a DNA molecule that is inversely complementary to the DNA molecule in a) or b) or c). The nucleotide sequence of the promoter comprises any one of 1)-3): 1) a DNA molecule with the nucleic acid sequence set forth in SEQ ID NO:3: 2) a genomic DNA molecule with 75% or more identity with the nucleotide sequence in 1): 3) a DNA molecule that is inversely complementary to the DNA molecule in 1) or 2).

In the present disclosure, there is no special restriction on the operation steps of the method that may cause the change in Ghpsm5 gene and/or promoter sequence, and those in conventional methods in the art may be used.

The present disclosure also provides a use of the RPGMS material prepared by the method in plant breeding.

In order to further explain the present disclosure, the RPGMS gene Ghpsm5 and the use thereof in cotton provided by the present disclosure will be described in detail in combination with the drawings and examples, but it should not be understood as limiting the protection scope of the present disclosure.

The experimental methods in the following examples are conventional methods unless otherwise specified.

The materials, reagents, etc. used in the following examples can be obtained commercially unless otherwise specified.

The public can obtain the Gossypium hirsutum CCIR 24 (Wang Xinyong, Liu Yining. The performance and cultivation techniques of CCIR 24 in northern Xinjiang [J]. China Cotton, 1998, 25 (1): 31-31.) in the following examples from the National Cotton Germplasm Medium Term Bank of the institute of cotton research of Chinese Academy Agricultural Sciences. The biological material is used only to repeat the relevant experiments of the present disclosure and cannot be used for other purposes.

The Agrobacterium tumefaciens LBA4404 in the following examples is a product of Beijing Dingguo Changsheng Biotechnology Co., Ltd., whose catalog number is MCC026.

### EXAMPLE 1

### Cotton RPGMS Material Obtained by Editing Ghpsm5 Gene Via Gene Editing Technology

### 1. Construction of Editing Vector

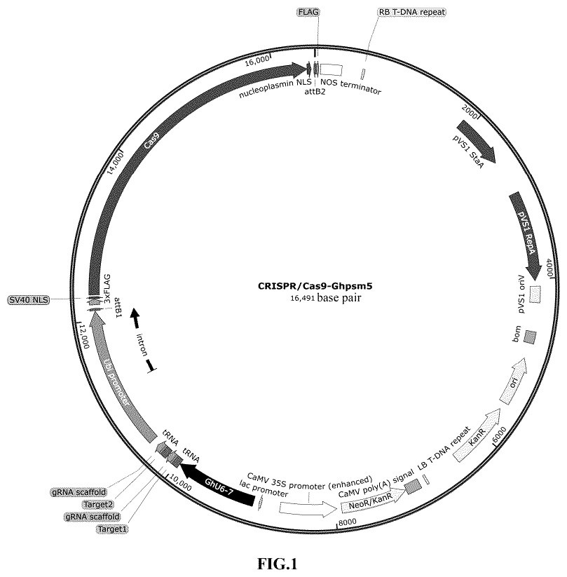

DNA fragment containing 2 gRNAs was ligated with a linear CRISPR/Cas9 plasmid obtained by BSAI enzyme digestion to obtain a ligated product. The ligated product was transformed into competent cells of Escherichia coli. Monoclones were picked up for positive detection to obtain a positive monoclone. Plasmids were extracted from the positive monoclone to obtain a CRISPR/Cas9-Ghpsm5 recombinant vector (FIG. 1).

gRNA:

SEQ ID NO: 4:
CCAAGCTCCCACATTAGAAA CGG
SEQ ID NO: 5:
CATTCCAGAAAGCAAACAAC AGG

Primers used for positive detection:

CAS9-F (SEQ ID NO: 6):
5′-ACACAGGAGCGTTTATATAAGCGA-3′,
CAS9-R (SEQ ID NO: 7):
5′-TGGTTTGTTGGTCGCCGTTAG-3′.

The reaction conditions for PCR amplification were: pre-denaturation at 94° C. for 5 min: 94° C. for 30 s, 58° C. for 30 s, 72° C. for 1 min, 30 cycles: and extension at 72° C. for 5 min.

### 2. Creation of the RPGMS Material by Gene Editing Technology

CRISPR/Cas9-Ghpsm5 recombinant vector was transformed into Agrobacterium tumefaciens LBA4404 to obtain a recombinant Agrobacterium tumefaciens containing CRISPR/Cas9-Ghpsm5, namely LBA4404/Ghpsm5.

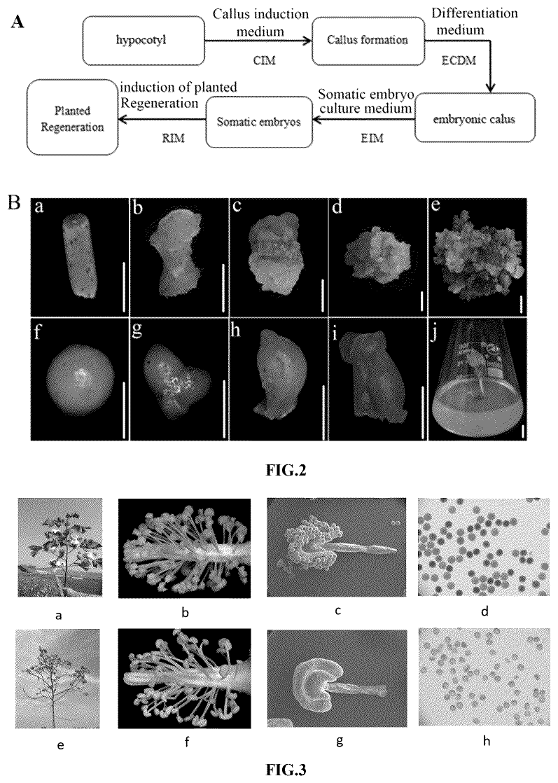

Agrobacterium-mediated genetic transformation was carried out according to the methods in the literature (Firoozabady E, DeBoer D L, Merlo D J, et al. Transformation of cotton (Gossypium hirsutum L.) by Agrobacterium tumefaciens and regeneration of transgenic plants [J]. Plant Molecular Biology, 1987, 10 (2): 105-116.). The hypocotyl of Gossypium hirsutum CCIR 24 was transformed by LBA4404/Ghpsm5 to transfer the gene to be edited into the cotton genome. Kanamycin was used for screen. Transgenic cotton was obtained after regeneration of the hypocotyl. The transgenic cotton was transplanted (FIG. 2). The T1 generation inbred seeds were harvested from T0 generation kanamycin-resistant plants. The molecular detection of PGMS individuals in T1 generation was carried out.

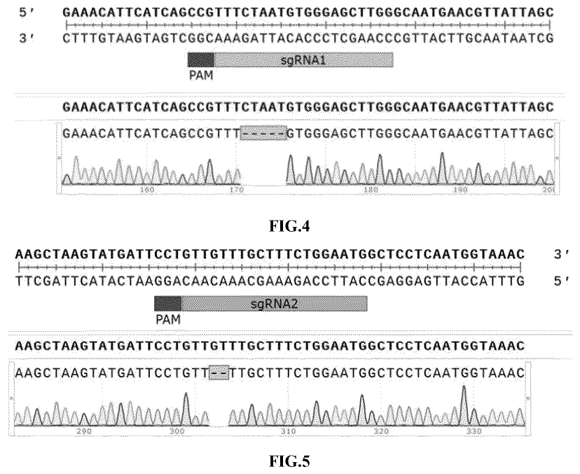

Leaves were taken to extract DNA. With the DNA as the template, and the following primers as primers, PCR amplification was carried out. The amplification product was recovered and connected to the T vector, which was then transferred into Escherichia coli. Monoclones were selected for sequencing to detect the gene editing. It was found after the sequencing that there were a 5-base deletion in target 1 segment and a 2-base deletion in target 2 segement of the Ghpsm5 gene in the RPGMS plant (The site sequence and detection results are shown in FIG. 4 and FIG. 5).

Ghpsm5-F1 (SEQ ID NO: 8):
5′-GGGCAGGATCGGAGATTGTT-3′
Ghpsm5-R1 (SEQ ID NO: 9):
5′-GCATCGGAACCCAACAGGA-3′
Ghpsm5-F2 (SEQ ID NO: 10):
5′-GGTTCTCGGTTGTTGCCAAT-3′
Ghpsm5-R2 (SEQ ID NO: 11):
5′-AGATAGGACAGCTACACAGGC-3′

The gene-edited material was planted continuously. It was found that the PGMS characteristic was maintained from T1 generation to T3 generation. The gene-edited material was named as ps201 (FIG. 3).

### 3. Genetic Analysis of Fertility of Gene-Edited Male Sterile Material

Tested materials: The cross was carried out with the non-transgenic CCIR 24 as the male parent and the RPGMS material ps201 obtained by gene editing as the female parent. The F₁ generation was normal and fertile. The F₂ generation included RPGMS individuals. The proportion of fertile and RPGMS individuals was 3:1, which conformed to the genetic law of single recessive gene. It was found that the RPGMS trait and the gene editing site were co-segregated through molecular identification.

The method of molecular identification: F₂ generation was planted in the experimental field of the institute of cotton research of Chinese Academy Agricultural Sciences in Anyang. Male fertile plants and male sterile plants were identified after their flowering. Among 136 F₂ plants, 32 male sterile plants were identified. DNA of 32 male sterile plants was extracted separately, and then 23 of them were mixed equally to obtain a mixed sample. The mixed sample and the remaining 9 plants, in total 10 plants, were detected together. Three fertile plants in the field were taken to detect one editing site. The results showed that there were no unedited sequences detected in male sterile single plants and mixed samples. 30 normal fertile single plants were selected for detection, among which 17 heterozygous single plants with edited sequence and non-edited sequence and 13 single plants without gene editing were detected.

### 3.1 Specific Detection Methods Were as Follows

a: The leaves of F₂ generation plants planted in the field were taken back to the laboratory with ice box and stored in the refrigerator at −80° C. DNA was extracted from leaves by Cetyltrimethylammonium Bromide (CTAB) method.
b: The sequence of the two editing sites was amplified from DNA by using KOD high-fidelity enzyme. Bands with the same size as the target segment were recycled. The target fragment was linked to a T vector and transformed into Escherichia coli. The Escherichia coli was coated on Luria-Bertani (LB) solid medium plate containing kanamycin, and put upside down in a 37° C. incubator overnight (12-24 hours).
c: (1) Growing monoclones were observed and picked. Kana antibiotic was added to LB in the ratio of 1 ml: 1 μL in the ultra-clean bench and gently shake up. 300-500 μL of the above LB solution to the centrifuge tube. Then the monoclones were taken to the centrifuge tube with a pipette tip. The LB solution with the monoclones was shaken in a shaker at 37° C. for more than 3 hours for detection. The Escherichia coli with the same size as the target fragment was sequenced.
d: The sequencing result was compared with the target sequence to determine whether editing was done.

### 4. Corresponding Time of Photoperiod of Gene-Edited RPGMS Line

ps201 was planted in flowerpots during the normal growth period of cotton in Anyang, Henan Province. After the cotton entered the flowering period, it was observed that ps201 was male sterility, with the anthers not cracking and the pollens having no vitality. The flowerpots were moved into the room for shading treatment in the afternoon, and moved outdoor for normal lighting after 20 pm. 17 days after the flowerpots were removed, when, the plant with a light duration at less than 12.0 hours started to disperse pollens normally, and the plant with more than 12.5 hours did not disperse pollens. It was thus inferred that ps201 was male sterile when the sunshine duration was more than 12.5 hours, and fertile when the sunshine duration was less than 12.0 hours.

ps201 was planted in the south breeding base of the institute of cotton research of Chinese Academy Agricultural Sciences in Yacheng, Hainan Province in late October. ps201 was normally fertile at flowering in December. By the middle of April of the next year, after the southern winter breeding, the sunshine duration in Hainan began to be more than 12 hours. ps201 turned into male sterility after 17 days of sunshine. The basal anthers of a small number of flowers occasionally dispersed pollens. When the cotton was planted in Anyang, Henan Province, and the sunshine duration changed from 12.5 hours to 12 hours during the period from the middle to the end of September, dispersed pollens from a small amount of anther was observed. 17 days after the sunshine duration became less than 12 hours, the cotton began to disperse pollens and set bolls like normal cotton, showing male fertility characteristics.

### EXAMPLE 2

### RPGMS Lines Prepared From Gene Editing Material

1. The RPGMS plant obtained in Example 1 was planted in an area where the sunshine was less than 12.0 hours and self-pollinated, such that an RPGMS line was generated.
2. The RPGMS line material obtained from step 1 was hybridized with normal fertile material. The RPGMS material was isolated from the offspring. The offspring material was planted in an area with more than 12.5 hours of sunshine duration. New cotton material with RPGMS characteristics was selected from the offspring material.

### EXAMPLE 3

### Use of the RPGMS Line in Breeding

Normal expression of Ghpsm5 gene will be destroyed by gene editing technology, resulting in recessive photoperiod-sensitive genic male sterility. In view of the fact that the cotton regions in China and the world's major cotton producing countries have more than 12.5 hours of sunshine in the normal growth stage of cotton and the RPGMS materials are all male sterile, such materials could be used as female parent for hybrid seed production. In the winter of tropical regions of the world where cotton grows normally, the fertility of the RPGMS material could be restored by placing it in an area where the sunshine hours are less than 12 hours (such as Sanya City, Hainan Province, China) and through self-crossing. The normally planted cotton without gene editing (except male sterile materials caused by other reasons) show normal fertility, and could be used as a male parent to cross with an RPGMS material to prepare hybrid with normal fertility. Therefore, normally planted cotton could be used as restorer lines for hybrid seed production, and the hybrids could be used for cotton hybrid production and breeding.
1. The RPGMS line obtained in Example 1 or Example 2 was used to cross with normal fertile cotton to cultivate hybrids.
2. The RPGMS line obtained in Example 1 or Example 2 was used to cross with normal fertile cotton to select excellent fertile material in the hybrid offspring to cultivate new varieties. The excellent RPGMS material in hybrid progeny was bred to cultivate RPGMS lines.

### EXAMPLE 4

### Use of the RPGMS Line in Intelligent Breeding

1. The RPGMS line was cultivated by the method of Example 1-3 and DNA sequencing was performed.
2. The RPGMS line sequenced in step 1 was planted to investigate the agronomic traits.
3. The cross was carried out with the excellent line as the male parent and the RPGMS plant as the female parent. The agronomic traits of the male parent and F₁ were investigated, and DNA of the male parent was sequenced.
4. A breeding model was established based on the parent traits, DNA sequences and the traits of F1 generation to form a parent selection algorithm for RPGMS lines.
5. The candidate male parent material was sequenced. The traits of hybrid F1 of the material and the RPGMS line were predicted by model calculation. The combinations with poor prediction results were eliminated, and the combinations with heterosis were prepared. Sequencing of hybrid breeding was carried out, and the agronomic traits of F1 generation were investigated. Steps 3 and 4 were repeated and the results were fed back to the model to improve the model. An intelligent breeding approach based on the RPGMS lines was formed.

Although the above embodiments give a detailed description of the present disclosure, they are only part not all of embodiments of the present disclosure. Those skilled in the art can also obtain other embodiments according to these embodiments without an inventive step, which are within the protection scope of the present disclosure.

1.-7. (canceled)

8. A method for preparing an RPGMS plant material, comprising regulating and/or changing an activity and/or expression pattern of a cotton recessive photoperiod-sensitive genic male sterile (RPGMS) gene Ghpsm5,
wherein an amino acid sequence of a protein encoded by the RPGMS gene Ghpsm5 comprises the sequence set forth in SEQ ID NO:2, or a sequence that has more than 75% identity with the amino acid sequence set forth in SEQ ID NO:2, or
wherein a nucleotide sequence of the RPGMS gene Ghpsm5 comprises the nucleotide sequence set forth in SEQ ID NO:1, or a nucleotide sequence of a derivate protein with the function of regulating anther dehiscence obtained by replacing and/or deleting and/or adding one or more amino acid residues to the protein set forth in SEQ ID NO:2.

9. The method according to claim 8, wherein a method for the regulating and/or changing comprises one or more of gene editing, RNAi, antisense RNA and DNA methylation.

10. The method according to claim 8, wherein a method for the regulating and/or changing comprises constructing an expression vector by using a promoter through genetic engineering to express a protein, RNA and/or DNA sequence that can affect the development of a male organ,
wherein the nucleotide sequence of the promoter comprises any one of the following:
1) a DNA molecule with the nucleic acid sequence set forth in SEQ ID NO:3;
2) a genomic DNA molecule with 75% or more identity with the nucleotide sequence in 1); or
3) a DNA molecule that is inversely complementary to the DNA molecule in 1) or 2).

11. The method according to claim 10, wherein the male organ comprises anthers.

12. A method for preparing a cotton RPGMS material comprising editing a cotton RPGMS gene and/or a promoter that regulates the expression of the cotton RPGMS gene Ghpsm5 with gene editing technology, wherein the editing the cotton RPGMS gene Ghpsm5 and/or the promoter comprises:
A) ligating a DNA fragment containing gRNAs set forth in SEQ ID NO:1-4 with a CRISPR/Cas9 linear plasmid obtained by BSAI enzyme digestion to obtain a ligated product, transforming the ligated product into a competent cell of Escherichia coli, selecting a monoclone for positive detection to obtain a positive monoclone, and extracting a plasmid from the positive monoclone to obtain a CRISPR/Cas9-Ghpsm5 recombinant vector;
B) transforming the CRISPR/Cas9-Ghpsm5 recombinant vector into Agrobacterium tumefaciens to obtain a recombinant Agrobacterium tumefaciens containing CRISPR/Cas9-Ghpsm5; and
C) transforming Gossypium hirsutum through agrobacterium-mediated genetic transformation to transfer CRISPR/Cas9-Ghpsm5 into cotton genome, then obtaining transgenic cotton and transplanting the transgenic cotton,
wherein an amino acid sequence of a protein encoded by the RPGMS gene Ghpsm5 comprises the sequence set forth in SEQ ID NO:2, or a sequence that has more than 75% identity with the amino acid sequence set forth in SEQ ID NO:2, or
wherein a nucleotide sequence of the RPGMS gene Ghpsm5 comprises the nucleotide sequence set forth in SEQ ID NO:1, or a nucleotide sequence of a derivate protein with the function of regulating anther dehiscence obtained by replacing and/or deleting and/or adding one or more amino acid residues to the protein set forth in SEQ ID NO:2;
wherein the nucleotide sequence of the promoter comprises any one of 1)-3):
1) a DNA molecule with the nucleic acid sequence set forth in SEQ ID NO:3;
2) a genomic DNA molecule with 75% or more identity with the nucleotide sequence in 1); or
3) a DNA molecule that is inversely complementary to the DNA molecule in 1) or 2).

13. The method according to claim 12, further comprising harvesting T1 generation inbred seeds from kanamycin resistant plants of T0 generation after transplanting the transgenic cotton, and carrying out molecular detection of RPGMS individuals in T1 generation.

14. The method according to claim 13, wherein the molecular detection comprises: extracting DNA from leaves, taking the DNA as a template, using the sequences set forth in SEQ ID NO:8, SEQ ID NO:9, SEQ ID NO:10 and SEQ ID NO: 11 as primers, performing PCR amplification, recovering an amplification product, ligating the amplification product to a T vector, transferring a T vector ligated to the amplification product into Escherichia coli, selecting a monoclone for sequencing, and detecting gene editing,
wherein an editing target 1 segment of the Ghpsm5 gene in RPGMS plant has 5 bases deletion and a target 2 segment has 2 bases deletion.

15. The method according to claim 12, wherein the sequence of primers used for the positive detection is set forth in SEQ ID NO:6 and SEQ ID NO:7.

16. The method according to claim 12, wherein procedures of the PCR amplification for positive detection comprise: pre-denaturation at 94° C. for 5 min; 94° C. for 30 s, 58° C. for 30 s, 72° C. for 1 min, 30 cycles; and extension at 72° C. for 5 min.

17. (canceled)

18. A method for cultivating an RPGMS line from the cotton RPGMS material prepared by the method according to claim 12, comprising the following steps:
planting the cotton RPGMS material in an area where the sunshine duration is less than 12.0 hours to obtain the RPGMS line after self-pollination and propagation; and
hybridizing the RPGMS line with a normal fertile material, and isolating an RPGMS material from a progeny, planting the RPGMS material from the progeny in an area where the sunshine duration is more than 12.5 hours, selecting a new cotton material with RPGMS characteristics from the RPGMS material from the progeny.

19. The method according to claim 8, wherein a nucleotide sequence of the RPGMS gene Ghpsm5 comprises any one of a)-d):
a) a DNA molecule or cDNA molecule encoded by SEQ ID NO:1;
b) a cDNA molecule or genomic DNA molecule encoding the protein set forth in SEQ ID NO:2 and having 75% or more identity with the nucleotide sequence set forth in SEQ ID NO:1;
c) a cDNA molecule or genomic DNA molecule encoded by SEQ ID NO:1 that Hybridizes with the nucleotide sequence of SEQ ID NO:1 under strict conditions; or
d) a DNA molecule that is inversely complementary to the DNA molecule in a) or b) or c).

20. The method according to claim 8, wherein regulating and/or changing comprises constructing an expression vector by using a promoter through genetic engineering to express a protein, RNA and/or DNA sequence that can affect the development of a male organ,
wherein the nucleotide sequence of the promoter is set forth in SEQ ID NO:3.

21. The method according to claim 12, wherein a nucleotide sequence of the RPGMS gene Ghpsm5 comprises any one of a)-d):
a) a DNA molecule or cDNA molecule encoded by SEQ ID NO:1;
b) a cDNA molecule or genomic DNA molecule encoding the protein set forth in SEQ ID NO:2 and having 75% or more identity with the nucleotide sequence set forth in SEQ ID NO:1;
c) a cDNA molecule or genomic DNA molecule encoded by SEQ ID NO:1 that Hybridizes with the nucleotide sequence of SEQ ID NO:1 under strict conditions; or
d) a DNA molecule that is inversely complementary to the DNA molecule in a) or b) or c).

22. The method according to claim 18, further comprising harvesting T1 generation inbred seeds from kanamycin resistant plants of T0 generation after transplanting the transgenic cotton, and carrying out molecular detection of RPGMS individuals in T1 generation.

23. The method according to claim 22, wherein the molecular detection comprises:
extracting DNA from leaves;
taking the DNA as a template;
using the sequences set forth in SEQ ID NO:8, SEQ ID NO:9, SEQ ID NO: 10 and SEQ ID NO:11 as primers;
performing PCR amplification;
recovering an amplification product;
ligating the amplification product to a T vector;
transferring a T vector ligated to the amplification product into Escherichia coli;
selecting a monoclone for sequencing; and
detecting gene editing,
wherein an editing target 1 segment of the Ghpsm5 gene in RPGMS plant has 5 bases deletion and a target 2 segment has 2 bases deletion.

24. The method according to claim 22, wherein the sequence of primers used for the positive detection is set forth in SEQ ID NO:6 and SEQ ID NO:7.

25. The method according to claim 22, wherein procedures of the PCR amplification for positive detection comprises:
pre-denaturation at 94° C. for 5 min, 94° C. for 30 s, 58° C. for 30 s, 72° C. for 1 min, or 30 cycles; and
extension at 72° C. for 5 min.

26. The method according to claim 8, wherein under a condition that a sunshine duration is greater than or equal to 12.5 hours, pollens of the RPGMS plant material are inactive and have no anther dehiscence, resulting in male sterility,
wherein under a condition that the sunshine duration is greater than 12.0 hours but less than 12.5 hours, anthers close to the base of flowers may crack and disperse pollens with normal vitality but small numbers, which make it difficult for the cotton to self-cross and set bolls, but may still be used for hybrid seed production, or
wherein under a condition that the sunshine duration is less than or equal to 12.0 hours, pollen development and anther dehiscence are normal, and male fertility is restored.

27. The method according to claim 12, wherein the nucleotide sequence of the promoter is set forth in SEQ ID NO:3.
